(19) United States
(12) Patent Application Publication
(10) Pub. No.: US 2019/0126075 A1
(43) Pub. Date: May 2, 2019

(54) FOOD AND DRINK PRODUCT CONTAINING POORLY DIGESTIBLE COMPOUND AND COLONIC-HYDROGEN-GAS PRODUCING AGENT

(71) Applicant: Kyodo Milk Industry Co., Ltd., Tokyo (JP)

(72) Inventors: Mitsuharu Matsumoto, Tokyo (JP); Ayako Fujita, Tokyo (JP); Kazuo Tsubota, Tokyo (JP)

(73) Assignee: Kyodo Milk Industry Co., Ltd., Tokyo (JP)

(21) Appl. No.: 15/764,577

(22) PCT Filed: Sep. 30, 2016
(86) PCT No.: PCT/JP2016/079102

§ 371 Date: Mar. 29, 2018

(30) Foreign Application Priority Data
Sep. 30, 2015 (JP) 2015-192937

(51) Int. Cl. A61P 39/06; A23L 33/135; A23C 9/12; A23L 2/52; A23L 33/125; A23L 33/24; A61K 35/745; A61K 31/7004; A61K 31/702; A61K 9/00

(52) U.S. Cl. CPC A61P 39/06; A23L 33/135; A23C 9/12; A23L 2/52; A23L 33/125; A61K 2035/115; A61K 35/745; A61K 31/7004; A61K 31/702; A61K 9/0095; A23V 2002/00; A23L 33/24

(57) ABSTRACT

The present invention provides foods and beverages comprising a combination of two or more digestion-resistant components. This invention also provides an agent for producing hydrogen gas in the large intestine, the agent comprising a combination of two or more digestion-resistant components. The foods and beverages or the agent for producing H₂ in the large intestine according to this invention can induce sustained production of large amounts of hydrogen gas by intestinal bacteria of more human individuals.

### TECHNICAL FIELD

The present invention relates to foods and beverages comprising a combination of two or more types of digestion-resistant components. This invention also relates to an agent for producing hydrogen gas in the large intestine, the agent comprising a combination of two or more types of digestion-resistant components.

### BACKGROUND ART

In 2007, it was reported that molecular hydrogen can selectively reduce hydroxy radicals, highly reactive active oxygen species, and protect cells from oxidative stress, and that hydrogen gas (H₂), when inhaled by rats, can suppress ischemia-reperfusion injury in rat brain (NPL 1). Since then, many studies have been conducted on the treatment and prophylaxis based on the antioxidant activity of H₂. Further, it was reported not only in animal studies but also in clinical studies that H₂ can mitigate type II diabetes (NPL 2) and ameliorate skin damage (NPL 3).

The currently prevailing method for supplying H₂ to the body is drinking hydrogen water, but the effect of hydrogen water supply lasts only transiently, for example for only about one hour. In contrast, the use of H₂ produced by degradation of digestion-resistant components by intestinal bacteria in the large intestine can be expected to allow for sustained supply of large amounts of H₂ to the body (NPLs 4, 5). For example, it was reported that milk-derived lactose (NPL 5), turmeric (NPL 6), fructan (NPL 7 and PTL 1), and pectin (NPL 8) are capable of inducing H₂ production by intestinal bacteria.

However, it is believed that the intestinal microbiota differs greatly among individuals, and thus, that the types of components that can induce H₂ production vary from individual to individual. In the meantime, there has been no study conducted to search for a digestion-resistant component that can induce H₂ production in intestinal microbiota of more human individuals.

### CITATION LIST

### Patent Literature

PTL 1: Japanese Patent Application Publication No. JP 2014-124100

### Non-Patent Literatures

NPL 1: Ohsawa, I., et al., Nat. Med., 2007, 13(6): p. 688-694.
NPL 2: Kajiyama, S., et al., Nutr. Res., 2008. 28(3): p. 137-143.
NPL 3: Li, Q., et al., Med. Gas. Res., 2013, 3(1): p. 20.
NPL 4: Suzuki, Y., et al., FEBS Lett., 2009, 583(13): p. 2157-2159.
NPL 5: Shimouchi, A., et al., Biomark. Insights, 2009, 4: p. 27-32.
NPL 6: Shimouchi, A., et al., Dig. Dis. Sci., 2009, 54(8): p. 1725-1729.
NPL 7: Nishimura, N., et al., J. Nutr., 2013, 143(12): p. 1943-1949.
NPL 8: Nishimura, N., et al., Br. J. Nutr., 2012, 107(4): p. 485-492.

### SUMMARY OF INVENTION

### Technical Problem

The present invention provides foods and beverages comprising a combination of two or more types of digestion-resistant components. This invention also provides an agent for producing H₂ in the large intestine, the agent comprising a combination of two or more types of digestion-resistant components.

### Solution to Problem

In consideration of the foregoing, the present inventors undertook a study focusing on H₂ generated by intestinal bacteria as a potential source of H₂ supply to the body. As a result of intensive studies, the inventors discovered particular combinations of digestion-resistant components. On the basis of this finding, the present invention has been completed.

More specifically, in certain embodiments, the present invention can be directed to the following.

[1] A food or beverage comprising a combination of two or more digestion-resistant components selected from the group consisting of maltitol, galactooligosaccharide, glucomannan, isomaltooligosaccharide, lactose, cellobiose, xylooligosaccharide, xylitol, sorbitol, erythritol, mannitol, pectin, resistant starch, resistant dextrin, and reduced resistant dextrin.
[2] The food or beverage as set forth in [1], wherein the combination of digestion-resistant components is:
(a) a combination of two digestion-resistant components selected from the group consisting of maltitol, galactooligosaccharide, and glucomannan; or
(b) a combination of maltitol, galactooligosaccharide, and glucomannan.
[3] The food or beverage as set forth in [1] or [2], wherein the combination of digestion-resistant components is present at a concentration of 0.01% w/w to 20% w/w.
[4] The food or beverage as set forth in any one of [1] to [3], further comprising a probiotic microorganism.
[5] The food or beverage as set forth in any one of [1] to [4], wherein the food or beverage is intended for use in producing hydrogen gas in the large intestine.
[6] The food or beverage as set forth in any one of [1] to [5], wherein the food or beverage is a dairy product.
[7] The food or beverage as set forth in [6], wherein the dairy product is selected from the group consisting of milk beverage, yogurt, ice cream, and pudding.
[8] An agent for producing hydrogen gas in the large intestine, the agent comprising a combination of two or more digestion-resistant components selected from the group consisting of isomaltooligosaccharide, lactose, galactooligosaccharide, cellobiose, xylooligosaccharide, xylitol, sorbitol, erythritol, maltitol, mannitol, pectin, glucomannan, resistant starch, resistant dextrin, and reduced resistant dextrin.
[9] The agent as set forth in [8], wherein the combination of digestion-resistant components is:
(a) a combination of two digestion-resistant components selected from the group consisting of maltitol, galactooligosaccharide, and glucomannan; or
(b) maltitol, galactooligosaccharide, and glucomannan.
[10] The agent as set forth in [8] or [9], further comprising a probiotic microorganism.

### Advantageous Effect of Invention

The foods and beverages or the agent for producing H₂ in the large intestine according to the present invention are useful as products that can induce sustained production of large amounts of H₂ through the use of intestinal bacteria of human individuals, despite the fact that human intestinal microbiota differs greatly among individuals.

### BRIEF DESCRIPTIONS OF DRAWINGS

FIG. 1 is a graph showing the averages for the concentrations of H₂ produced in the fecal incubation system in the presence of maltitol, galactooligosaccharide, glucomannan, or a mixture of these three digestion-resistant components.

FIG. 2 is a set of graphs showing the percentages of fecal samples in which the concentration of H₂ produced in the fecal incubation system in the presence of maltitol, galactooligosaccharide, glucomannan, or a mixture of these three digestion-resistant components was enhanced by at least three times relative to the control.

FIG. 3 is a graph showing change in the averages for H₂ concentrations in breath samples from 6 fasted subjects fed with 200 mL of maltitol/galactooligosaccharide/glucomannan-supplemented milk, normal cows' milk, or water.

FIG. 4 is a graph showing the averages for H₂ concentrations in the gases generated in the bodies of LKM512-treated or PBS (control)-treated ICR (male) mice before and after administration of digestion-resistant component-supplemented milk.

FIG. 5 is a graph showing change in the averages for H₂ concentrations in breath samples from 7 fasted subjects fed with 200 mL of a maltitol/galactooligosaccharide/glucomannan-supplemented milk beverage, normal cows' milk, or hydrogen water.

### DESCRIPTION OF EMBODIMENTS

The following provides specific descriptions of the present invention, but this invention is not limited thereto. Unless otherwise defined herein, scientific and technical terminologies used in connection with this invention shall have meanings generally understood among skilled artisans.

(Foods and Beverages)

The present invention relates to a food or beverage comprising two or more types of digestion-resistant components.

As referred to herein, the two or more types of digestion-resistant components are a combination of two or more type of digestion-resistant components selected from the group consisting of maltitol, galactooligosaccharide, glucomannan, isomaltooligosaccharide, lactose, cellobiose, xylooligosaccharide, xylitol, sorbitol, erythritol, mannitol, pectin, resistant starch, resistant dextrin, and reduced resistant dextrin. Preferably, the two or more types of digestion-resistant components are a combination of two members selected from the group consisting of maltitol, galactooligosaccharide, and glucomannan, or a combination of three members consisting of maltitol, galactooligosaccharide, and glucomannan.

The food or beverage of the present invention may comprise not only the aforementioned two or more types of digestion-resistant components but also any other components that are known to be capable of inducing H₂ production by intestinal bacteria. Examples of such other components include lactose, turmeric, fructan, lactulose, raffinose, soya oligosaccharides, lactosucrose, chitosan oligosaccharides, cyclic oligosaccharides, and palatinose.

Maltitol is 4-O-α-D-glucopyranosyl-D-glucitol, and is also called reduced malt sugar.

Galactooligosaccharide is 4′-galactosyllactose.

Glucomannan is a polysaccharide composed of glucose and mannose at a ratio of 2:3, which are linked in a linear fashion by β-1,4-bonds. The degree of polymerization of glucomannan used in the present invention is not particularly limited.

Isomaltooligosaccharide is a polysaccharide composed of glucose building blocks and having one or more α-1,6-, α-1,4- and α-1,3-bonds. The degree of polymerization of isomaltooligosaccharide used in the present invention is not particularly limited. Preferred examples of isomaltooligosaccharide used in this invention include isomaltose, isomaltotriose, and panose.

Xylooligosaccharide is a polysaccharide composed of about 2 to 7 xylose units linked by β-1,4-bonds.

Pectin is a complex polysaccharide consisting mainly of polygalacturonic acid, which is composed of galacturonic acid units linked by α-1,4-bonds. The molecular weight of pectin that can be used in the present invention is not particularly limited.

Resistant starch (RS) is a generic name that refers to a starch or starch degradation products which reach the large intestine without being digested in human digestive system up to the small intestine. RS is classified into four types according to the characteristics. Type 1 (RS1) is a starch that is not acted on by a digestive enzyme like α-amylase, as found in coarsely milled grains; type 2 (RS2) is a starch with high amylose content; type 3 (RS3) is a retrograded starch which is structurally modified during a process in which a starch gelatinized by heat cooking, etc. cools down; and type 4 (RS4) is a processed starch (chemically modified starch). In the present invention, any type of resistant starch can be used.

Resistant dextrin is a dietary fiber obtained by hydrolyzing a starch in the presence of trace acid at high temperature, further hydrolyzing it with α-amylase and glucoamylase, and then purifying the resulting hydrolysate. Resistant dextrin, which is a glucan with an average molecular weight of about 2,000, contains not only α-1,4- and α-1,6-bonds inherently present in starch but also other bonds like α-1,2- and α-1,3-bonds, and so has a more highly branched structure than the starting starch. Reduced resistant dextrin is a reduced product of resistant dextrin.

The concentration of the combination of digestion-resistant components to be added to the food or beverage of the present invention is not particularly limited, but the combination of digestion-resistant components is present at a concentration of preferably 0.01% w/w to 20% w/w, more preferably 0.5% w/w to 15% w/w, much more preferably 1% w/w to 10% w/w, relative to the weight of the food or beverage of this invention.

The proportions of different digestion-resistant components to be added are not particularly limited and can be determined by a skilled artisan as appropriate. For example, different digestion-resistant components are each preferably present in a proportion of at least 0.5%, at least 1%, at least 5%, at least 10%, or at least 20%, relative to the total amount of digestion-resistant components. Different digestion-resistant components can be present in equal amounts or in different proportions, relative to the total amount of the digestion-resistant components.

For example, when the combination of digestion-resistant components comprises maltitol, galactooligosaccharide, and glucomannan, the concentration of maltitol present in the food or beverage can be in the range of 0.01% w/w to 3% w/w, preferably 0.5% w/w to 2.5% w/w, or 0.8% w/w to 1.5% w/w; the concentration of galactooligosaccharide present in the food or beverage can be in the range of 0.01% w/w to 3% w/w, preferably 0.5% w/w to 2.5% w/w, or 0.8% w/w to 1.5% w/w; and the concentration of glucomannan present in the food or beverage can be in the range of 0.01% w/w to 2.5% w/w, preferably 0.05% w/w to 2.0% w/w, or 0.05% w/w to 0.5% w/w. It should be noted that the aforementioned concentrations are relative to the weight of the food or beverage.

The food or beverage of the present invention may comprise not only the aforementioned two or more types of digestion-resistant components but also a probiotic microorganism. As referred to herein, the “probiotic microorganism” refers to a live microorganism that, when ingested by a human individual, can offer benefit to the individual. The probiotic microorganism that may be present in the food or beverage of this invention is not particularly limited, and can be exemplified by microorganisms belonging to the genus Bifidobacterium which are designated as Lactobacillus bifidus in the art, and microorganisms regarded as lactic acid bacteria, such as Lactobacillus genus microorganisms, Lactococcus lactis, Enterococcus faecalis, and Pediococcus pentosaceus. The probiotic microorganism may comprise such a single strain of microorganism or may comprise a combination of such two or more species or strains of microorganisms.

Examples of microorganisms belonging to the genus Lactobacillus include, but are not limited to, Lactobacillus casei, Lactobacillus plantarum, Lactobacillus brevis, Lactobacillus rhamnosus, Lactobacillus gasseri, Lactobacillus delbrueckii, Lactobacillus helveticus, Lactobacillus paracasei, Lactobacillus acidophilus, and Lactobacillus reuteri.

Examples of microorganisms belonging to the genus Bifidobacterium include Bifidobacterium animalis subsp. animalis, Bifidobacterium animalis subsp. lactis, Bifidobacterium pseudocatenulatum, Bifidobacterium catenulatum, Bifidobacterium bifidum, Bifidobacterium longum, Bifidobacterium breve, Bifidobacterium infantis, and Bifidobacterium adolescentis. Among them, preferably used are Bifidobacterium animalis subsp. lactis and Bifidobacterium pseudocatenulatum, and more preferably used is Bifidobacterium animalis subsp. lactis. In one mode, the Bifidobacterium animalis subsp. lactis strain LKM512 can be used. The LKM512 strain is available from the depository institution (International Patent Organism Depositary (IPOD), National Institute of Technology and Evaluation (NITE)) under Accession No. FERMP-21998.

The amount of the probiotic microorganism present in the food or beverage of the present invention is not particularly limited. For example, the food or beverage can be formulated to ensure that the probiotic microorganism is present in an amount of 2×10³ to 8×10¹² cfu, preferably 2×10⁵ to 8×10¹¹ cfu, more preferably 2×10⁷ to 8×10¹⁰ cfu, per 100 g of the food or beverage. As referred to herein, the “cfu” refers to colony-forming units. The cfu value can be measured using any method known to skilled artisans, for example, by counting viable colonies grown after a solution of a microorganism diluted with phosphate buffer solution (PBS) is seeded on MRS medium and incubated at 37° C. for 48 hours.

The type of the food or beverage of the present invention is not particularly limited as long as it is a food or beverage, and the food or beverage is preferably a dairy product or a Western fresh confectionary. The type of the dairy product is not particularly limited as long as it is a food or beverage produced using raw or processed milk as an ingredient, and examples of the dairy product include milk beverage, cheese, fermented milk, and ice cream. Examples of the European fresh confectionary include pudding. In a particularly preferred mode, the food or beverage of this invention is a milk beverage.

As referred to herein, the “milk beverage” refers to a beverage produced by adding a non-milk-derived component to raw or processed milk used as an ingredient. The milk beverage may further comprise skimmed milk (a product obtained by hydrating skimmed milk powder) or skimmed milk powder. The milk beverage may also contain a further additive, as exemplified by a substance for making adaptation to the preferences for taste, aroma, etc., such as coffee, fruit juice or flavorant, or by a nutrient such as vitamins or minerals.

The food or beverage of the present invention, when ingested by a subject, can act on intestinal bacteria in the large intestine of the subject, thereby inducing H₂ production. Therefore, the food or beverage of this invention can be used for producing H₂ in the large intestine. The effect of the food or beverage of this invention to induce H₂ production in the large intestine can be confirmed by, for example, such a method as described below in the Examples section. To be specific, this effect can be confirmed by the following procedure: a breath sample is collected from a subject ingesting the food or beverage of this invention at a specified point of time after ingestion of the food or beverage, and the H₂ concentration in the collected sample is compared to that in a breath sample collected from the same subject before ingestion of the food or beverage. Alternatively, this effect can also be confirmed by comparing the H₂ concentration in a breath sample collected from a subject ingesting the food or beverage of this invention after a lapse of a specified time, to that in a breath sample collected from the same subject ingesting a (control) food or beverage not supplemented with the inventive combination of digestion-resistant components, after a lapse of the same specified time.

(Agent for Producing H₂ in the Large Intestine)

The present invention relates to an agent for producing H₂ in the large intestine, the agent comprising a combination of two or more types of digestion-resistant components. The combination of two or more types of digestion-resistant components present in the agent for producing H₂ in the large intestine according to this invention is as defined above in the section describing the food or beverage of this invention.

The amount of the combination of digestion-resistant components to be added to the agent for producing H₂ in the large intestine according to the present invention is not particularly limited. The agent is formulated to ensure that the combination of digestion-resistant components is contained in an amount of preferably 0.1 g to 20 g, more preferably 0.5 g to 20 g, much more preferably 1 g to 10 g, per dose of the agent.

The proportions of different digestion-resistant components to be added are not particularly limited and can be determined by a skilled artisan as appropriate. For example, different digestion-resistant components are each preferably present in a proportion of at least 0.5%, at least 1%, at least 5%, at least 10%, or at least 20%, relative to the total amount of digestion-resistant components. Different digestion-resistant components may be present in equal amounts or in different amounts, relative to the total amount of the digestion-resistant components.

For example, when the combination of digestion-resistant components comprises maltitol, galactooligosaccharide, and glucomannan, the amount of maltitol present in the agent for producing H₂ in the large intestine can be in the range of 0.02 g to 6.0 g, preferably 1.0 g to 5.0 g, or 1.5 g to 3.0 g; the amount of galactooligosaccharide present in the agent for producing H₂ in the large intestine can be in the range of 0.02 g to 6.0 g, preferably 1.0 g to 5.0 g, or 1.5 g to 3.0 g; and the amount of glucomannan present in the agent for producing H₂ in the large intestine can be in the range of 0.02 g to 5 g, preferably 0.05 g to 3.0 g, or 0.1 g to 1.0 g. It should be noted that the aforementioned amounts are per dose of the agent for producing H₂ in the large intestine.

The agent for producing H₂ in the large intestine according to the present invention may further comprise a probiotic microorganism. The probiotic microorganism that may be present in the agent for producing H₂ in the large intestine according to this invention is as defined above in the section describing the food or beverage of this invention. In this aspect of the invention, the agent for producing H₂ in the large intestine can be a single composition comprising both the combination of digestion-resistant components and the probiotic microorganism, or can be a combined composition consisting of a composition comprising the combination of digestion-resistant components and a composition comprising the probiotic microorganism. When the agent is such a combined composition, the composition comprising the combination of digestion-resistant components and the composition comprising the probiotic microorganism can be designed to be ingested concurrently, or can be designed to be ingested separately.

The amount of the probiotic microorganism present in the agent for producing H₂ in the large intestine according to the present invention is not particularly limited. For example, the agent for producing H₂ in the large intestine can be formulated to ensure that the probiotic microorganism is contained in an amount of 2×10³ to 8×10¹² cfu, preferably 2×10⁵ to 8×10¹¹ cfu, more preferably 2×10⁷ to 8×10¹⁰ cfu, per dose of the agent.

The form of the agent for producing H₂ in the large intestine according to the present invention is not particularly limited as long as it is in a form suitable for ingestion by humans. For example, the agent for producing H₂ in the large intestine can be in the form of liquid, suspension (liquid dispersion), emulsion, semi-solid, paste, powder, granule, tablet, tableted preparation, capsule or pill. The agent for producing H₂ in the large intestine according to this invention may further comprise an additive such as sweetener, antiseptic, colorant, antioxidant or flavorant.

The effect of the agent for producing H₂ in the large intestine according to the present invention can be confirmed by the method described above in the section describing the food or beverage of this invention.

(Method for Producing H₂ in the Large Intestine)

The present invention also relates to a method for producing H₂ in the large intestine of a subject, the method comprising the step of feeding or administering to the subject, the food or beverage or the agent for producing H₂ in the large intestine according to this invention.

The present invention also relates to a combination of two or more types of digestion-resistant components, or a food or beverage comprising said combination, as described above, which are intended for use in a method for producing H₂ in the large intestine of a subject. This invention further relates to use of two or more types of digestion-resistant components, as described above, in the production of an agent for producing H₂ in the large intestine.

As referred to herein, the “subject” refers to a mammalian animal, preferably a human.

While the present invention has hereinbefore been described in general, the following provides certain examples for reference to help get a better understanding of this invention. However, these examples are provided to illustrate this invention and not to limit this invention.

### EXAMPLES

### Example 1: Selection of Digestion-Resistant Components with High H₂ Production Promoting Activity (in Fecal Incubation System)

<Reagents>

Twenty-three different types of digestion-resistant components (cellulose, isomaltooligosaccharide, lactose, fructooligosaccharide, galactooligosaccharide, cellobiose, xylooligosaccharide, xylitol, sorbitol, erythritol, maltitol, mannitol, pectin, sodium alginate, glucomannan, lignin, chitin, chitosan, resistant starch, resistant dextrin, reduced resistant dextrin, kale, or young barley leaf) were each diluted with phosphate buffered saline (PBS) to adjust the concentration to 2.5% (w/v). Among these components, those which were not completely dissolved were used in a suspended state. The control used was a PBS solution not containing any of these components.

<Subjects, Fecal Samples>

Fecal samples were collected from 10 human healthy adult subjects (7 male, 3 female, average aged 35.8), and were used within 5 hours after defecation.

<Fecal Treatment and Incubation>

Twenty milliliters of an anaerobic dilution solution (a solution prepared by dissolving KH₂PO₄, Na₂HPO₄, L-cysteine hydrochloride, Tween 80, and 0.1% Resazurin in a vial, jet-injecting N₂ and CO₂ (80%, 20%) into the vial, and autoclaving the vial sealed with a butyl rubber stopper) was added to 5 g of each of the collected samples, and the mixture was stirred by vortexing and then centrifuged (at 1000 g for 1 min.) to remove solids such as undigested food debris. 160 μL of each of the fecal suspension supernatants harboring fecal bacteria was dispensed into an Eppendorf tube, and 40 μL of each of the prepared solutions of digestion-resistant components in PBS was added to each of the Eppendorf tubes (final concentrations of digestion-resistant components: 0.5%). The Eppendorf tubes containing a mixture of fecal suspension and digestion-resistant component were placed into vial bottles (40 mm×75 mm, 50 mL) while the lids of the tubes and bottles were left open, and a mixed gas of N₂ and CO₂ (79.8%, 20.2%) was jet-injected to render the Eppendorf tubes and the bottles anaerobic. The vials were sealed with butyl rubber stoppers and incubated in an incubator (at 37° C. for 24 hours). The experiments were done in duplicate per sample, with each experiment being repeated three times.

<H₂ Concentration Measurement>

Half a milliliter of the gas in each of the vial bottles was collected in a 1.0 mL gastight syringe (produced by Hamilton Company) and analyzed by TRIlyzer (Taiyo Nippon Sanso Corporation). In this process, when concentration exceeded the detection limit, 1 mL of the sample was injected into a 50 mL vial bottle, diluted 50-fold, and subjected to concentration measurement. Before gas collection, the gas in the vial bottles was stirred five times using the syringe.

<Results>

The ratio of the H₂ production concentration in each of the samples relative to that in the control was calculated, and those sample/control ratios of 10 or higher are shaded in the table (Table 1). Such a sample/control ratio of 10 or higher was observed in the fecal samples collected from 5 of 10 isomaltooligosaccharide-ingested subjects, 4 of 10 lactose-ingested subjects, 7 of 10 fructooligosaccharide-ingested subjects, 5 of 10 galactooligosaccharide-ingested subjects, 7 of 10 cellobiose-ingested subjects, 1 of 10 xylooligosaccharide-ingested subjects, 6 of 10 xylitol-ingested subjects, 4 of 10 sorbitol-ingested subjects, 2 of 10 erythritol-ingested subjects, 6 of 10 maltitol-ingested subjects, 5 of 10 mannitol-ingested subjects, 1 of 10 pectin-ingested subjects, 3 of 10 glucomannan-ingested subjects, 1 of 10 resistant starch-ingested subjects, 2 of 10 resistant dextrin-ingested subjects, and 2 of 10 reduced resistant dextrin-ingested subjects.

TABLE 1

| | | Subject No. | | | | | | | | | |
|---|---|---|---|---|---|---|---|---|---|---|---|
| | | 1 | 2 | 3 | 4 | 5 | 6 | 7 | 8 | 9 | 10 |
| Digestion-resistant component | Cellulose | 1.0 | 1.4 | 0.6 | 0.9 | 1.1 | 1.3 | 0.8 | 0.9 | 1.1 | 1.1 |
| | Isomaltooligosaccharide | 4.1 | 13.6* | 44.4* | 15.2* | 26.8* | 23.3* | 0.7 | 0.7 | 0.1 | 7.5 |
| | Lactose | 4.9 | 9.5 | 36.6* | 28.7* | 22.4* | 22.1* | 0.5 | 0.7 | 0.1 | 7.1 |
| | Fructooligosaccharide | 9.6 | 30.6* | 47.2* | 52.8* | 3.4 | 44.3* | 24.7* | 4.4 | 34.9* | 15.6* |
| | Galactooligosaccharide | 5.3 | 18.5* | 35.8* | 16.9* | 15.4* | 22.1* | 0.8 | 0.8 | 0.1 | 5.9 |
| | Cellobiose | 25.7* | 37.0* | 67.3* | 76.5* | 8.1 | 52.0* | 3.1 | 7.5 | 46.4* | 18.9* |
| | Xylooligosaccharide | 1.7 | 5.2 | 25.0* | 7.9 | 5.0 | 8.2 | 0.3 | 0.8 | 0.0 | 3.1 |
| | Xylitol | 18.4* | 11.1* | 40.2* | 72.4* | 8.8 | 3.2 | 1.4 | 1.9 | 12.5* | 60.2* |
| | Sorbitol | 31.6* | 9.6 | 33.0* | 54.8* | 3.4 | 4.2 | 68.6* | 1.7 | 0.1 | 4.5 |
| | Erythritol | 12.7* | 27.6* | 3.8 | 1.5 | 7.1 | 1.8 | 4.0 | 1.8 | 1.0 | 2.1 |
| | Maltitol | 12.5* | 7.2 | 386.6* | 22.1* | 2.6 | 8.4 | 315.5* | 0.8 | 55.6* | 32.3* |
| | Mannitol | 2.2 | 28.2* | 234.7* | 81.9* | 6.6 | 76.2* | 54.3* | 1.7 | 0.0 | 3.5 |
| | Pectin | 6.0 | 9.1 | 8.2 | 6.2 | 2.4 | 7.7 | 27.0* | 3.7 | 0.2 | 6.8 |
| | Sodium alginate | 1.1 | 2.7 | 2.3 | 7.8 | 1.8 | 3.1 | 1.3 | 1.3 | 1.0 | 2.2 |
| | Glucomannan | 1.8 | 22.9* | 1.6 | 1.9 | 7.6 | 8.2 | 3.2 | 39.1* | 20.4* | 7.3 |
| | Lignin | 0.3 | 2.0 | 0.8 | 1.5 | 0.1 | 1.3 | 1.1 | 1.3 | 2.0 | 1.3 |
| | Chitin | 1.2 | 1.9 | 0.6 | 1.4 | 0.6 | 1.6 | 1.1 | 0.8 | 1.1 | 1.1 |
| | Chitosan | 0.9 | 1.1 | 0.9 | 1.0 | 1.1 | 1.1 | 0.5 | 0.8 | 0.5 | 0.8 |
| | Resistant starch | 3.1 | 1.2 | 2.0 | 0.9 | 10.5* | 1.2 | 1.2 | 1.8 | 1.4 | 8.9 |
| | Resistant dextrin | 4.2 | 7.6 | 5.0 | 5.2 | 2.4 | 8.6 | 12.6* | 2.9 | 5.9 | 15.3* |
| | Reduced resistant | 6.8 | 7.6 | 8.0 | 8.1 | 3.3 | 7.7 | 24.1* | 2.2 | 7.1 | 29.6* |
| | dextrin | | | | | | | | | | |
| | Kale | 0.8 | 1.3 | 1.1 | 1.0 | 0.6 | 1.6 | 1.2 | 1.1 | 0.4 | 0.8 |
| | Young barley leaf | 0.9 | 0.9 | 1.0 | 1.2 | 0.6 | 1.5 | 1.3 | 0.5 | 1.0 | 1.1 |

values with *: sample/control ratios of ≥10

When each of the digestion-resistant components was ingested alone in such a manner, it was found that the types of the digestion-resistant components observed to exhibit H₂ production promoting activity varied among individuals. The reason for this is believed to be as follows: since the intestinal microbiota differs greatly among individuals, the types of the components that can be degraded in the large intestine also vary from human to human. Thus, we searched for a combination of digestion-resistant components (with a sample/control ratio of 10 or higher) which can be expected to exhibit H₂ production promoting activity in all subjects, and as a result, found that a combination of maltitol, galactooligosaccharide and glucomannan can take effect in all subjects (Table 2).

TABLE 2

| | Subject No. | | | | | | | | | |
|---|---|---|---|---|---|---|---|---|---|---|
| | 1 | 2 | 3 | 4 | 5 | 6 | 7 | 8 | 9 | 10 |
| Maltitol | 12.5* | 7.2 | 386.6* | 22.1* | 2.6 | 8.4 | 315.5* | 0.8 | 55.6* | 32.3* |
| Galactooligosaccharide | 5.3 | 18.5* | 35.8* | 16.9* | 15.4* | 22.1* | 0.8 | 0.8 | 0.1 | 5.9 |
| Glucomannan | 1.8 | 22.9* | 1.6 | 1.9 | 7.6 | 8.2 | 3.2 | 39.1* | 20.4* | 7.3 |

values with *: sample/control ratios of ≥10

### Example 2: Verification of the Synergistic Effect of Combining Digestion-Resistant Components (in Fecal Incubation System)

<Reagents>

Each of maltitol, galactooligosaccharide and glucomannan was dissolved in PBS to give a 0.8% solution. Similarly, in order to prepare a mixed solution of the three components, these components were mixed at a concentration of 0.8% each, and dissolved in PBS. The control used was a PBS solution not containing any of these components.

<Subjects, Fecal Samples>

Fecal samples from 14 human healthy adult subjects (11 male, 3 female, average aged 40.9) were used.

<Fecal Treatment and Incubation, and H₂ Concentration Measurement>

Fecal treatment and incubation, and H₂ concentration measurement, were performed by the same procedures as in Example 1.

<Results>

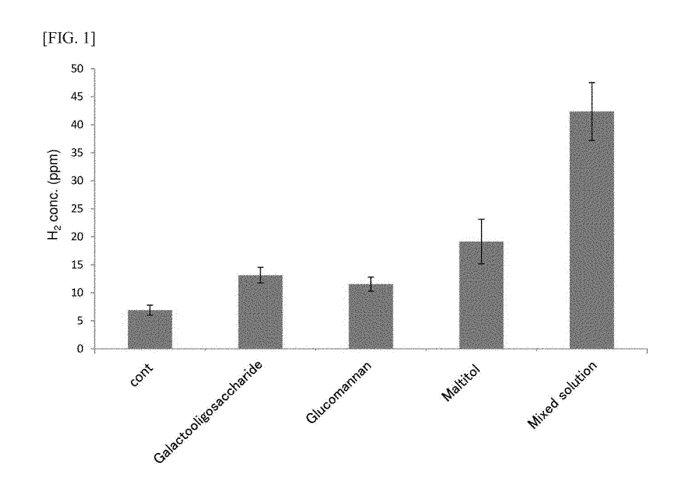

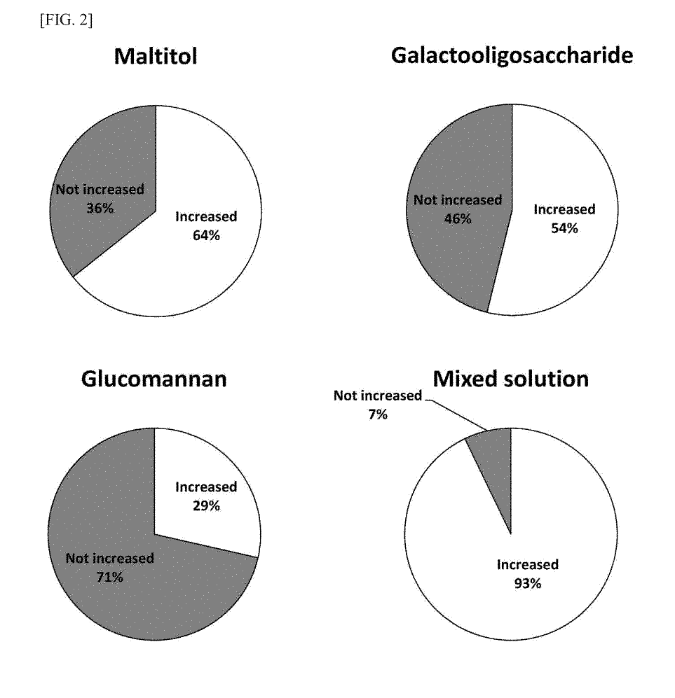

The concentration of H₂ produced increased in the case of ingestion of a mixture of the three components than in the case of ingestion of each of these components alone (FIG. 1). A sample/control H₂ production concentration ratio of 2 or higher was observed in the fecal samples from 9 of 14 subjects ingesting maltitol alone, 8 of 14 subjects ingesting galactooligosaccharide alone, and 4 of 14 subjects ingesting glucomannan alone, whereas such a sample/control ratio of 2 or higher was observed in the fecal samples from 13 of 14 subjects ingesting a mixture of the three components (FIG. 2). Likewise, a sample/control H₂ production concentration ratio of 3 or higher was observed in the fecal samples from 5 of 14 maltitol-ingested subjects, 0 of 14 galactooligosaccharide-ingested subjects, and 1 of 14 glucomannan-ingested subjects, but was observed in the fecal samples from 11 of 14 subjects ingesting a mixture of the three components. Further, a sample/control H₂ production concentration ratio of 5 or higher was observed only in the fecal samples from 2 of 14 maltitol-ingested subjects, and not in any of the fecal samples from the galactooligosaccharide- or glucomannan-ingested subjects, but even such a level of ratio was observed in the fecal samples from 9 of 14 subjects ingesting a mixture of the three components. The results given above demonstrated that combining these three digestion-resistant components produces a wide coverage of H₂ production promoting activity.

### Example 3: Investigation of H₂ Producing Milk Beverage (Measurement of Breath H₂ Concentration) (1)

<Subjects>

The subjects used were 6 human healthy adults with no risk of developing diarrhea after drinking milk.

<Test Foods>

A total of three different test foods, as presented below, were used for comparison: 200 mL of water (control); 200 mL of milk; and digestion-resistant component-supplemented milk (prepared by dissolving 2 g each of maltitol, galactooligosaccharide and glucomannan in 200 mL of milk).

<Schedule for Breath Sample Collection>

After fasted for 12 hours (with ad libitum access to water only), the subjects were asked to ingest 200 mL of milk over one minute. On different days, the same test was conducted in the subjects who were asked to ingest a different test food, i.e., water (control) or digestion-resistant component-supplemented milk. In consideration of a burden on the health conditions of the subjects, the tests were conducted at intervals of 2 days or longer. Breath samples were collected every one hour for 12 hours after ingestion of the food. A breath sample was also collected immediately before ingestion of the test food, and regarded as a sample collected at 0 hour after test food ingestion.

<Procedure for Breath Sample Collection>

Before breath sample collection, the subjects were asked to seat themselves quietly for 5 minutes. Breath sample collection was done through a series of actions to be performed by the subjects, as mentioned below: after a subject seats him/herself for 5 minutes quietly, s/he breathes in deeply through the nose, holds breath for 15 seconds, and exhales slowly, and then further expires his/her end-alveolar breath (i.e., end expiration) into a breath collection bag (produced by Otsuka Pharmaceutical Co., Ltd.).

<Measurement of H₂ Concentration in Breath Sample>

After the breath sample collected in each breath collection bag was stirred five times using a gastight syringe, 0.5 mL of the breath sample in the bag was drawn in the syringe. The breath samples thus drawn in the syringes were measured for H₂ concentration using the biogas measurement system TRIlyzer (Taiyo Nippon Sanso Corporation).

<Results>

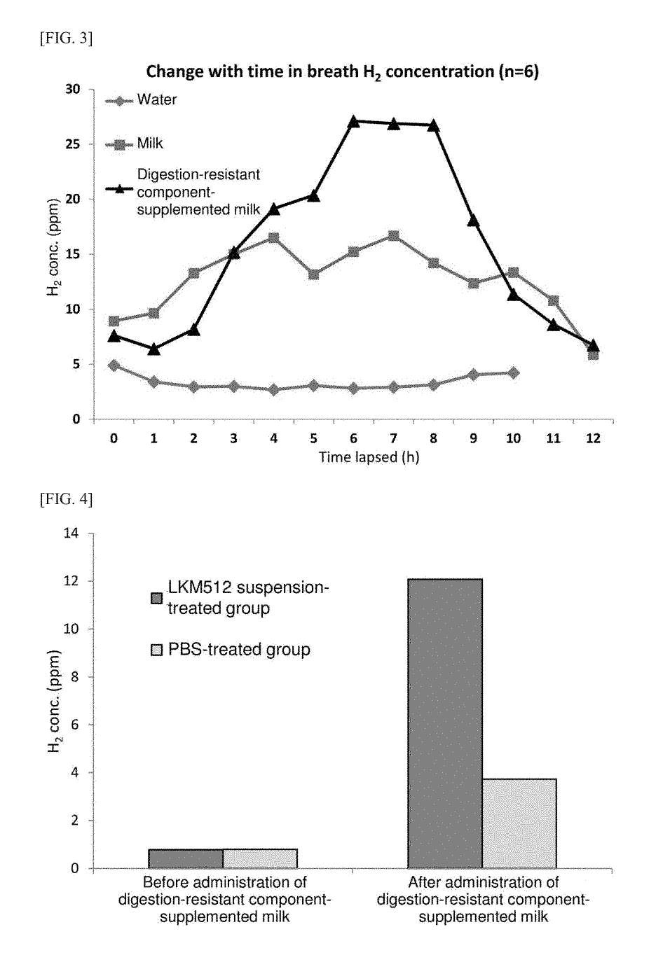

The H₂ concentration in breath increased in the case of ingestion of digestion-resistant component-supplemented milk than in the case of ingestion of milk alone (FIG. 3). The analysis of AUC (area under the curve) values also demonstrated that digestion-resistant component-supplemented milk had high H₂ production promoting activity.

### Example 4: Investigation of Addition of a Probiotic Microorganism to a H₂ Producing Milk Beverage (Measurement of Mouse Breath H₂ Concentration)

<Test Animals>

Ten ICR (male) mice aged 9 weeks were divided into two groups (n=5): the group treated with a suspension of the Bifidobacterium animalis subsp. lactis strain LKM512 (hereinafter also referred to simply as “LKM512”), and the PBS-treated group (control). The LKM512 suspension or PBS was orally administered forcedly to the mice using a stomach sonde at a dose of 300 μL/day five times a week for 3 weeks. The number of LKM512 strains in the LKM512 suspension was 7.0×10⁸ cfu/300 μL. After completion of the dosing period of 3 weeks, the mice were fed with digestion-resistant component-supplemented milk (prepared in the same way as in Example 3) on day 21, and were subjected to measurement of H₂ production concentration.

<Measurement of H₂ Production Concentration>

After fasted for 15 hours, the mice were placed in holding cages (with no feed, water or bedding) for 2 hours and acclimated to the environment. Then, 360 μL of digestion-resistant component-supplemented milk was orally administered forcedly to the mice. The measurement was taken twice each, before and 2 hour after the administration of digestion-resistant component-supplemented milk, and two measurements taken at each time point were averaged.

<Sample Collection and Measurement Procedures>

The mice were each locked for 5 minutes in an acrylic chamber sealed with a butyl rubber stopper with an air collection port, to thereby ensure that the gases (breath and flatus gases) generated from the mouse body accumulated in the chamber. Then, 0.5 mL of the air in the chamber was collected from the port of the stopper using a gastight syringe. In this process, the air in the chamber was stirred by turning on a fan mounted in the chamber. The collected air samples were measured for H₂ concentration using the biogas measurement system TRIlyzer (Taiyo Nippon Sanso Corporation).

<Results>

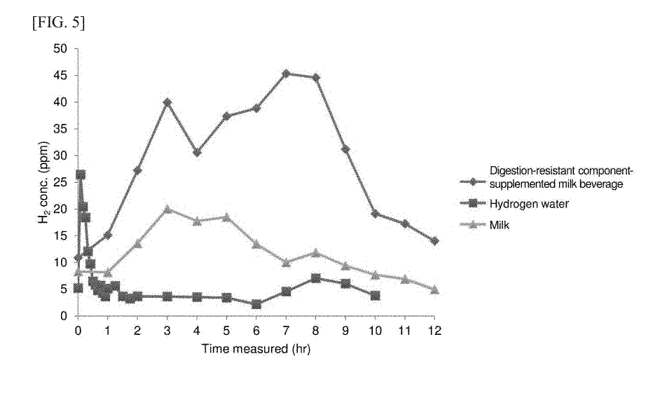

No difference in H₂ production concentration was found between the two groups before administration of digestion-resistant component-supplemented milk, but after administration of digestion-resistant component-supplemented milk, the H₂ production concentration was about 3.2 times higher in the LKM512 suspension-treated group than in the PBS-treated group (FIG. 4).

### Example 5: Investigation of H₂ Producing Milk Beverage (Measurement of Breath H₂ Concentration) (2)

<Test Procedure>

A single-arm, intra-group comparative, open-label study was conducted in 7 human healthy adults with no risk of developing diarrhea after drinking milk. The test foods used were a digestion-resistant component-supplemented milk beverage (prepared by adding digestion-resistant components to a milk beverage containing raw milk and skimmed milk powder, so as to give galactooligosaccharide, maltitol and glucomannan concentrations of 1.6%, 1.1% and 0.1%, respectively), normal cows' milk, and commercial hydrogen water (produced by Melodian Co. Ltd.). Measurement was performed by observing hospitalized subjects throughout the day. The subjects were hospitalized a total of three times, once a week. The subjects were asked to ingest 200 mL of a digestion-resistant component-supplemented milk beverage during first hospitalization, 200 mL of normal cows' milk during second hospitalization, and 200 mL of hydrogen water during third hospitalization, whereby the influences of the test foods on breath H₂ concentration were compared.

After fasting for 12 hours (with ad libitum access to water only), the subjects ingested 200 mL of a digestion-resistant component-supplemented milk beverage, normal cows' milk, or hydrogen water—this time point was regarded as a baseline for the test. The subjects further ingested 100 mL of OS-1 (produced by Otsuka Pharmaceutical Co., Ltd.) every 2 hours from 2 hours after the start of the study (i.e., after 2, 4, 6, 8 and 10 hours), and also ingested a Boncolon diet for breakfast (produced by Otsuka Foods Co., Ltd.) after 4 hours. Ingestion of water was prohibited after the start of the study. In the case of ingestion of digestion-resistant component-supplemented milk beverage or normal cows' milk, measurement of breath H₂ concentration was taken a total of 13 times, first immediately before the start of the study, and then every one hour until 12 hours later. In the case of ingestion of hydrogen water, measurement of breath H₂ concentration was taken first immediately before the start of the study, and then every 5 minutes in the first hour, every 15 minutes from one hour later to 2 hours later, and every one hour from 2 hours later to 10 hours later. Collection of breath samples and measurement of H₂ concentration in the breath samples were performed by following the procedures described above in Example 3.

<Results>

The breath H₂ concentration increased more significantly in the case of ingestion of the digestion-resistant component-supplemented milk beverage than in the case of ingestion of normal cows' milk, and this result demonstrated the effectiveness of the supplementary components (FIG. 5). Also, according to comparison with commercial hydrogen water, the breath H₂ concentration for the first 20 minutes after ingestion was higher in the case of hydrogen water ingestion, but the breath H₂ concentration for the subsequent periods was predominantly higher in the case of ingestion of the digestion-resistant component-supplemented milk beverage. The analysis of AUC (area under the curve) values also showed that the AUC value of the digestion-resistant component-supplemented milk beverage was 2.5 and 6.7 times higher than those of normal cows' milk and hydrogen water, respectively.

1. A food or beverage comprising a combination of two or more digestion-resistant components selected from the group consisting of maltitol, galactooligosaccharide, glucomannan, isomaltooligosaccharide, lactose, cellobiose, xylooligosaccharide, xylitol, sorbitol, erythritol, mannitol, pectin, resistant starch, resistant dextrin, and reduced resistant dextrin.

2. The food or beverage according to claim 1, wherein the combination of digestion-resistant components is:
(a) a combination of two digestion-resistant components selected from the group consisting of maltitol, galactooligosaccharide, and glucomannan; or
(b) a combination of maltitol, galactooligosaccharide, and glucomannan.

3. The food or beverage according to claim 1, wherein the combination of digestion-resistant components is present at a concentration of 0.01% w/w to 20% w/w.

4. The food or beverage according to claim 1, further comprising a probiotic microorganism.

5. (canceled)

6. The food or beverage according to claim 1, wherein the food or beverage is a dairy product.

7. The food or beverage according to claim 6, wherein the dairy product is selected from the group consisting of milk beverage, cheese, fermented milk, and ice cream.

8. (canceled)

9. (canceled)

10. (canceled)

11. A method for producing hydrogen gas in the large intestine, comprising administering to a subject in need thereof an effective amount of a combination of two or more digestion-resistant components selected from the group consisting of isomaltooligosaccharide, lactose, galactooligosaccharide, cellobiose, xylooligosaccharide, xylitol, sorbitol, erythritol, maltitol, mannitol, pectin, glucomannan, resistant starch, resistant dextrin, and reduced resistant dextrin.

12. The method according to claim 11, wherein the combination of digestion-resistant components is:
(a) a combination of two digestion-resistant components selected from the group consisting of maltitol, galactooligosaccharide, and glucomannan; or
(b) maltitol, galactooligosaccharide, and glucomannan.

13. The method according to claim 11, further comprising administering an effective amount of a probiotic microorganism to the subject.

14. The method according to claim 11, wherein the combination is administered as a food or beverage as defined in claim 1.
